(19) United States
(12) Patent Application Publication
(10) Pub. No.: US 2012/0329133 A1
(43) Pub. Date: Dec. 27, 2012

(54) PROCESS FOR PURIFYING HEPARAN-N-SULFATASE

(76) Inventor: David Nichols, Newton, MA (US)

(21) Appl. No.: 13/475,568

(22) Filed: May 18, 2012

Related U.S. Application Data
(60) Provisional application No. 61/488,090, filed on May 19, 2011.

(51) Int. Cl. C12N 9/14

(52) U.S. Cl. 435/195

(57) ABSTRACT

A process for preparing and purifying heparan-N-sulfatase is disclosed involving chromatographic steps for producing or purifying heparan-N-sulfatase under conditions that yield highly pure heparan-N-sulfatase.

### RELATED APPLICATIONS

This application claims the benefit of U.S. Provisional Application Ser. No. 61/488,090, filed May 19, 2011, the entire teachings of which are incorporated herein by reference.

### FIELD OF THE INVENTION

The present inventions relate to processes and methods for preparing and purifying heparan-N-sulfatase. The disclosed processes and methods generally comprise subjecting heparan-N-sulfatase to one or more chromatographic steps under conditions that yield highly pure heparan-N-sulfatase.

### BACKGROUND

The mucopolysaccharidoses (MPS) are a group of rare, inherited lysosomal storage disorders caused by the deficiency or absence of specific lysosomal enzymes. The absence of these enzymes results in the accumulation of complex sugar molecules in the cells and tissues, as well as in cellular organelles called lysosomes. In the presence of normal lysosomal enzymes these sugars are transformed into other substances and used by the body. These complex sugars are known as mucopolysaccharides or glycosaminoglycans (GAGs) and serve as the building blocks for connective tissues in the body.

MPS III results from the lack of four different enzymes necessary to degrade the GAG. Each enzyme deficiency defines a different form of Sanfilippo syndrome: type IIIA (Sanfilippo A), type IIIB (Sanfilippo B), type IIIC (Sanfilippo C), and type IIID (Sanfilippo D). Heparan-N-sulfatase (HNS) is an enzyme that participates in the stepwise degradation of heparan sulfate. HNS hydrolyzes the sulfate moiety attached to the amino group of the glucosamine residue of heparan sulfate, a type of GAG. A deficiency of this enzyme is associated with mucopolysaccharidoses IIIA (MPS, Sanfilippo's syndrome A). Patients affected by MPS type III A have mutations in the gene coding for HNS, resulting in a deficiency or absence of this enzyme.

Symptoms of MPS IIIA (Sanfilippo A) usually arise between 2 to 6 years of age, although in some cases diagnosis is made as late as 13 years of age. The clinical symptoms of the condition present with differing degrees of severity. The central nervous system is the most severely affected system in patients with MPS IIIA. HNS and other secondarily stored compounds accumulate primarily in the central nervous system. Problems in language development, motor skills, and intellectual development characterize the condition. Overall, individuals with MPS IIIA have a marked developmental delay, and long-term survival is poor. The condition is chronically debilitating and life-threatening.

Presently no approved therapeutic treatments for MPS IIIA are available. Bone marrow transplant has been used in an attempt to slow disease progression. Because heparan sulfate is the natural substrate of HNS, animal studies have shown that HNS may be useful for the treatment of lysosomal storage disorders, such as MPS IIIA, in which there is an increase in heparan sulfate.

Given the interest in HNS as a pharmaceutical agent, there remains a need for preparation of large quantities of highly purified material in a cost effective manner. Various reports of purifying HNS from culture medium have been reported (Hemsley et al., Mol. Genet. Metab. 90:313-328 (2007)). While several methods of purification of HNS have been attempted and described, none have been suitable for production of HNS for use in human therapy of MPS IIIA.

### SUMMARY

Embodiments provided herein relate generally to a process for the purification of heparan-N-sulfatase (HNS), in particular the purification of recombinant human HNS (rhHNS) from culture medium or a semi-purified sample of crude recombinant HNS, as well as to compositions and formulations comprising HNS purified by the process and methods of using said purified HNS. Described methods comprise the use of a combination of chromatographic methods to purify HNS.

Embodiments described herein are based on the recognition that the published procedures for isolating HNS do not reproducibly yield HNS of sufficient purity and solubility to be therapeutically useful. Based on this recognition, both methods and assays are provided herein for reproducibly preparing HNS under conditions that reduce levels of contaminants. Producing and purifying HNS by these methods provides HNS that contains reduced amounts of contaminants. In some embodiments, the HNS obtained by the methods and assays provided herein yields HNS that contains reduced amounts of high pI HNS which may reduce its solubility. The purification process allows for large amount of higher yields of HNS and higher purity of HNS than that provided by known processes. This purification process is particularly useful for preparing pharmaceutical grade HNS for use in humans (e.g., rhHNS).

In one aspect, methods and assays are provided herein for purifying recombinant human HNS by purifying material extracted from cell culture medium and exposing the extracted material to one or more column chromatography or batch chromatography media (e.g., one, two, three, four, five, six, seven, eight, nine, ten or more). Similarly, in one aspect, methods and assays and provided herein for purifying recombinant human HNS by further purifying an enriched eluate extracted from one or more column chromatography or batch chromatography media by exposing such enriched eluate to one or more additional column chromatography or batch chromatography purification steps (e.g., one, two, three, four, five, six, seven, eight, nine, ten or more).

In one embodiment, HNS is purified using a four column process comprising the purification steps of 1) filtering the initial HNS in solution extracted from the cell culture medium; 2) loading the filtered HNS onto an anion exchange matrix (e.g., Q Sepharose Fast Flow™ column), washing the column and eluting the enriched HNS from the column; 3) loading the eluate from the anion exchange column onto a hydrophobic interaction column (HIC) (e.g., Phenyl Sepharose), washing the column and eluting the enriched HNS from the column; 4) loading the eluate from the HIC column onto a hydroxyapatite column (HA) (e.g., ceramic hydroxyapatite Type 1), washing the column and eluting the enriched HNS from the column; and 5) loading the eluate from the HA column onto a cationic exchange column (e.g., SP Sepharose), washing the column and eluting the enriched HNS from the column. In certain embodiments, the eluate recovered from the cationic exchange column is further filtered and concentrated by ultrafiltration or diafiltration. In certain embodiments, the eluate recovered from the cationic exchange column is further purified (e.g., by loading such eluate into one or more of the anionic exchange column, the HIC column, the HA column and/or the cationic exchange column).

It should be noted that in certain embodiments, the performance of each of the column chromatography purification steps need not necessarily be dependant on the previously performed column chromatography purification step. Accordingly, in certain embodiments the order in which each of the column chromatography purification steps are performed is not critical, and references made in a subsequent column chromatography purification step to, for example, a specific eluate from a previous step, are made for convenience and/or clarity. For example, in certain embodiments, HNS is purified using a four column process, however after filtering the initial HNS in solution extracted from the cell culture medium the recited purification steps are performed in a different order. For example, the purification steps may comprise 1) loading the filtered HNS onto a anion exchange matrix (e.g., Q Sepharose Fast Flow™ column), washing the column and eluting the enriched HNS from the column; 2) loading the eluate from the anion exchange column onto a hydroxyapatite (HA) column (e.g., ceramic hydroxyapatite Type 1), washing the column and eluting the enriched HNS from the column; 3) loading the eluate from the HA column onto a cationic exchange column (e.g., SP Sepharose), washing the column and eluting the enriched HNS from the column; and 4) loading the eluate from the cationic exchange column onto a hydrophobic interaction column (HIC) (e.g., Phenyl Sepharose), washing the column and eluting the enriched HNS from the column).

In certain embodiments, HNS is purified using at least one, at least two, at least three, at least four or more column chromatography purification steps. For example, in certain embodiments, HNS is purified using a three column process wherein after filtering the initial HNS in the solution extracted from the cell culture medium, such filtered HNS is subjected to purification comprising the steps of 1) loading the HNS onto a anion exchange matrix (e.g., Q Sepharose Fast Flow™ column), washing the column and eluting the enriched HNS from the column; 2) loading the eluate from the anion exchange column onto a hydrophobic interaction column (HID) (e.g., Phenyl Sepharose), washing the column and eluting the enriched HNS from the column; and 3) loading the eluate from the HIC column onto a hydroxyapatite column (HA) (e.g., ceramic hydroxyapatite Type 1), washing the column and eluting the enriched HNS from the column.

Similarly, in certain embodiments, HNS is purified using at least two column chromatography steps. For example, in certain embodiments, following filtering of the initial HNS from solution extracted from the cell culture medium, such HNS is purified using a two column process comprising the purification steps of 1) loading the HNS onto a hydrophobic interaction column (HIC) (e.g., Phenyl Sepharose), washing the column and eluting the enriched HNS from the column; and 2) loading the eluate from the HIC column onto a hydroxyapatite column (HA) (e.g., ceramic hydroxyapatite Type 2), washing the column and eluting the enriched HNS from the column.

While each of the recited column chromatography purification steps may be performed without respect to a previous column chromatography purification step, it should be understood that the individual components which comprise each of the column chromatography purification steps are intended to be performed in the order recited. For example, the step of loading an initial HNS extract (or alternatively an eluate from a previously performed column chromatography purification step) onto a column must precede washing of that column, and washing of the column must precede the elution of the enriched HNS from that column.

In accordance with a further aspect provided herein, a purified recombinant HNS composition is described that can be useful for treating a subject suffering from a lysosomal enzyme deficiency such as MPS IIIA. HNS has been shown to have activity when administered via the cerebrospinal fluid in a naturally occurring mouse model of MPS IIIA. Hemsley, et al., Mol. Genet. Metab. 90:313-328 (2007). Intra-cisternal delivery of HNS in a MPS IIIA Huntaway dog model also showed therapeutic activity. Hemsley, et al., Mol. Genet. Metab. 98(4): 383-92 (2009).

In accordance with yet another aspect provided herein, compositions of HNS are described that are substantially free of high pI HNS. In another aspect, the disclosure provides compositions of HNS that are substantially pure HNS. In certain embodiments, the purified HNS preparation is greater than about 90% free of contaminants. Preferably, the material is greater than 80%, 85%, 90%, 91%, 92%, 93%, 94%, 95%, 96%, 97%, 98%, or even greater than 99% free of contaminants.

In accordance with another aspect, a pharmaceutical composition is provided herein that comprises a therapeutically effective amount of purified recombinant HNS as prepared by the process described herein, together with suitable excipients. The pharmaceutical composition of recombinant HNS is particularly suitable for topical, oral or parenteral (e.g., intravenous, subcutaneous, intramuscular or intrathecal) administration to a subject.

The above discussed and many other features and attendant advantages of the present invention will become better understood by reference to the following detailed description of the invention when taken in conjunction with the accompanying examples. The various embodiments described herein are complimentary and can be combined or used together in a manner understood by the skilled person in view of the teachings contained herein.

### BRIEF DESCRIPTION OF THE DRAWINGS

FIG. 1 illustrates a purification process flow diagram of an embodiment provided herein.

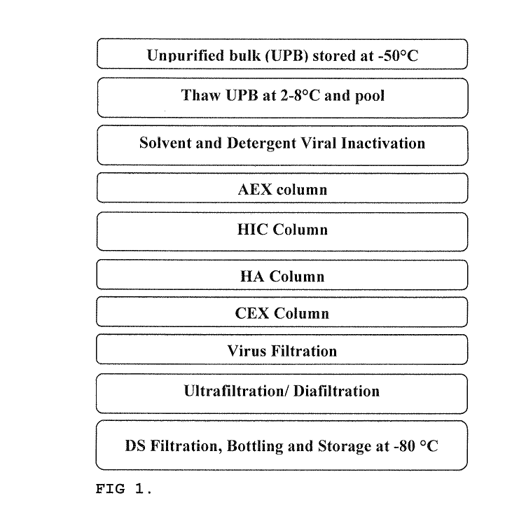

### DETAILED DESCRIPTION

Certain embodiments described herein provide methods and processes for preparing purified HNS, a lysosomal enzyme for use in the treatment of MPS IIIA. Certain embodiments described herein provide methods of treating a subject (e.g., a subject with MPS IIIA) with the purified HNS compositions disclosed herein. Processes for purifying HNS are known in the art. See e.g., Hemsley et al., Mol. Genet. Metab. 90:313-328 (2007); U.S. Patent Application Pub. No. 2009/0186011 each of which is incorporated herein by reference. However, previously published methods for preparing HNS were not manufacturable, were not easily scaled up and/or do not reproducibly yield pure HNS suitable for use in humans.

Producing and purifying HNS according to methods disclosed herein provides HNS that contains reduced amounts of contaminants. The HNS produced by methods described herein is particularly well suited for use as a therapeutic agent (e.g., for the treatment of MPS IIIA).

Heparan-N-sulfatase is a lysosomal enzyme also known in the art by the names N-sulphoglucosamine sulphohydrolase; SGSH; EC 3.10.1.1; N-sulfoglucosamine sulfohydrolase; 2-desoxy-D-glucoside-2-sulphamate sulphohydrolase (sulphamate sulphohydrolase); heparin sulfamidase; sulfoglucosamine sulfamidase; sulphamidase; HNS, rhHNS, sulfamidase, rhNS, and rhSGSH. The term “HNS” as used herein encompasses this enzyme, including functional fragments and/or derivatives thereof, and any pharmaceutically acceptable forms thereof. Heparin-N-sulfatase is associated with Online Mendelian Inheritance in Man (OMIM) identification no. OMIM 605270, the entry for which is publicly available online at http://www.ncbi.nlm.nih.gov/omim/605270. The entire contents of this online entry, and all pages linked thereon, are herein incorporated by reference.

As used herein, “HNS composition” means any composition containing HNS, in various states of purity.

As used herein, the term “substantially pure” means that the proteins or polypeptides are essentially free of other substances to an extent practical and appropriate for their intended use. In particular, the proteins are sufficiently pure and are sufficiently free from other biological constituents of their hosts cells and viruses so as to be useful in, for example, pharmaceutical preparations. As used herein, a “substantially pure HNS” is a preparation of HNS, which has been isolated or synthesized and which is greater than about 90% free of contaminants. Preferably, the material is greater than 80%, 85%, 90%, 91%, 92%, 93%, 94%, 95%, 96%, 97%, 98%, or even greater than 99% free of contaminants. The degree of purity may be assessed by means known in the art.

The terms “treat” and “treating” as used herein refer to reversing or blocking the progression of the disease in the subject.

Because HNS is a naturally occurring enzyme, it is typically prepared by isolation from a cell culture supernatant medium obtained from a host cell suitable for making the protein. In certain embodiments the host cell is genetically engineered to produce HNS. For example, the genes responsible for the cellular machinery that produce HNS can be placed into a microorganism such as bacteria or fungi. In other embodiments, the genes responsible for the cellular machinery that produce HNS can be placed into a mammalian cell. Non-limiting examples of mammalian cells that may be used include BALB/c mouse myeloma line (NSO/l, ECACC No: 85110503); human retinoblasts (PER.C6, CruCell, Leiden, The Netherlands); monkey kidney CV1 line transformed by SV40 (COS-7, ATCC CRL 1651); human embryonic kidney line (293 or 293 cells subcloned for growth in suspension culture, Graham et al., J. Gen Virol., 36:59, 1977); human fibrosarcoma cell line (HT1080); baby hamster kidney cells (BHK, ATCC CCL 10); Chinese hamster ovary cells+/−DHFR(CHO, Urlaub and Chasin, Proc. Natl. Acad. Sci. USA, 77:4216, 1980); mouse sertoli cells (TM4, Mather, Biol. Reprod., 23:243-251, 1980); monkey kidney cells (CV1 ATCC CCL 70); African green monkey kidney cells (VERO-76, ATCC CRL-1 587); human cervical carcinoma cells (HeLa, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci., 383:44-68, 1982); MRC 5 cells; FS4 cells; and a human hepatoma line (Hep G2).

In certain aspects provided herein, the culture conditions for the host cells are optimized to produce a high level of HNS with minimal levels of contaminants. In another aspect provided herein, the process of purifying HNS is intended for use with biological materials, particularly crude mixtures containing HNS and other contaminating proteins, referred to as starting material samples or bulk material. In accordance with one aspect provided herein, a method for the purification of HNS is described, in particular for the purification of recombinant human HNS (rhHNS), from a crude preparation of the culture medium of the recombinant process or bulk material. The rhHNS obtained by this method has a high degree of purity and high specific bioactivity (e.g., in the range of at least 10 units/mg, at least 15 units/mg, at least 20 units/mg, at least 25 units/mg, at least 30 units/mg, at least 35 units/mg, at least 40 units/mg, at least 45 units/mg, at least 47 units/mg, at least 50 units/mg, at least 60 units/mg, at least 70 units/mg, at least 75 units/mg, at least 85 units/mg, at least 90 units/mg, at least 100 units/mg, or more), and is practically free from host cell proteins which are present in the culture medium and from nucleic acids or other contaminants contained in the host cells used in the recombinant process.

In one embodiment, the sample of HNS is initially constituted by collecting cell culture supernatant medium. It is contemplated that the crude solution may be filtered or concentrated and subjected to one or more steps to remove contaminants derived from the cell culture to yield bulk material. The purification process as described herein may include one or more subsequent chromatography steps (e.g., at least one, at least two, at least three, at least four, at least five, at least six, at least seven, at least eight, at least nine, at least ten, or more chromatography steps) in order to achieve a desired degree of purity of HNS.

In certain embodiments the semi-purified material is first captured by exposure to mercapto-ethyl-pyridine. In one embodiment the mercapto-ethyl-pyridine is 4-mercapto-ethyl-pyridine linked to a cellulose matrix.

In another embodiment, the HNS material is subjected to viral inactivation prior to being further purified. Viral inactivation may be accomplished, for example by adding 1% Tween 80 and 0.3% TnBP to in-process HNS samples or media and holding at ambient temperature for 3-16 hours. This step may be performed at any point in the purification scheme. Further, filtration of the HNS composition using a 0.2 μm filter may be incorporated into any loading step.

In yet other embodiments, the resulting HNS material is optionally reduced in volume prior to column chromatography purification. In other embodiments, the volume is reduced following recovery of the enriched HNS eluate following the column chromatography steps.

In certain embodiments, methods and processes for purifying HNS by a sequence of chromatography steps are included. In certain embodiments, the performance of each of the disclosed column chromatography purification steps need not necessarily be performed. Similarly, to the extent the multiple column chromatography steps are disclosed, such steps need not be performed sequentially or in the recited order. For example, in certain embodiments, the HNS is purified using at least one, at least two, at least three, at least four or more column chromatography purification steps. Similarly, in certain embodiments one or more of the recited column chromatography steps may be performed multiple times. In some embodiments, one or more of the chromatography steps includes loading, equilibrating, washing, and eluting of the chromatography medium or resin. Notwithstanding the foregoing statements regarding the sequential performance of each of the chromatography purification steps, it should be understood that the individual components which comprise each of the column chromatography purification steps are intended be performed in the order recited. For example, as will be appreciated by one of skill in the art, the steps of loading, equilibrating, washing, and eluting which generally comprise each chromatography purification step are intended to be performed in the recited order.

Exemplary purification techniques include batch chromatography and column chromatography. In some embodiments a HNS composition is contacted with a series of chromatographic media during purification. In certain embodiments, the chromatography media or resins include one or more anionic exchange resin. In another embodiment, the chromatography media or resin includes one or more hydrophobic interaction resin. In yet another embodiment, the chromatography media or resin includes one or more hydroxyapatite resin. In other embodiments, the chromatography media or resin includes one or more cationic exchange resin.

In certain embodiments, the chromatography media or resins include an anionic exchange resin, a hydrophobic interaction resin, a hydroxyapatite resin, and a cationic exchange resin. In certain embodiments, the extracted material is purified using a column packed with Q Sepharose, followed by a column packed with Phenyl Sepharose, followed by a column packed with ceramic hydroxyapatite Type I; and finally followed by another column packed with SP Sepharose. The contemplated steps for purifying the extracted material need not all be performed. For example, in certain embodiments the extracted material is purified using a column packed with Q Sepharose, followed by a column packed with Phenyl Sepharose, followed by a column packed with ceramic hydroxyapatite Type I. Similarly, the contemplated steps for purifying the extracted material need not all be performed in any particular order. For example, in certain embodiments, the extracted material is purified using a column packed with Q Sepharose, followed by a column packed with Phenyl Sepharose, followed by another column packed with SP Sepharose; finally followed by a column packed with ceramic hydroxyapatite Type I. In each of the forgoing embodiments, each of the columns is optionally washed with buffered or other aqueous solution followed by elution of HNS using an aqueous solution. In certain embodiments, the HNS composition is eluted from the chromatography medium between each step. It is contemplated that each elution step may be repeated one or more times before advancing to the next purification step. In certain embodiments the extracted material is further purified by filtration. In yet other embodiments, the extracted material is subjected to viral inactivation before, after or during chromatography.

In one embodiment, the chromatography media or resins comprise an anionic exchange resin. In certain embodiments, contacting the HNS composition with the anionic exchange chromatography resin is, for example, the first, second, third or fourth chromatographic step. Various chromatographic resin or medium may be employed, including, for example, resins from GE HealthCare, Tosoh Biosciences, Applied Biosystems, Bio-Rad, and Pall. Examples of suitable anionic exchange chromatography media are diethylaminoethyl (DEAE), quaternary aminoethyl (QAE) or quaternary ammonium (O) resin. In certain embodiments, the anionic exchange chromatography resin is a Q sepharose fast flow resin.

In another embodiment, the process of purification of HNS comprises a hydrophobic interaction chromatography (HIC) step. In some embodiments, the HNS composition is contacted with a hydrophobic interaction chromatography resin as an intermediate step in the purification process. In other embodiments, contacting the HNS composition with the hydrophobic interaction chromatography resin is, for example, the first, second, third or fourth chromatographic step. Examples of suitable hydrophobic interaction chromatography media include phenyl, octyl, butyl, hexyl, propyl, PPG, or ether. In certain embodiments, purification of the HNS extract is performed using a Phenyl Sepharose 6 Fast Flow column. In certain embodiments, the HNS composition or eluate resulting from contact with the hydrophobic interaction chromatography resin is further contacted with a hydroxyapatite chromatography resin.

In yet another embodiment, the chromatography media or resin comprises a hydroxyapatite (HA) resin. In other embodiments, contacting the HNS composition with the hydroxyapatite resin is, for example, the first, second, third or fourth chromatographic step. In some embodiments, the extract containing HNS is purified using a column packed with ceramic hydroxyapatite Type I. In some embodiments, the extract containing HNS is purified using a column packed with ceramic hydroxyapatite Type II. In yet another embodiment the HNS composition or eluate collected from the interaction with the hydroxyapatite chromatography resin is further contacted with a cationic exchange chromatography resin.

In certain embodiments, the HNS composition is further purified using a cationic exchange chromatography step. In certain embodiments, the purification using a cationic exchange chromatography step is an intermediate step in the purification of HNS. In other embodiments, contacting the HNS composition with the cationic exchange chromatography resin is the first, second, third, forth or last chromatographic step. In some embodiments, the chromatography media or resin comprises a cationic exchange resin. Examples of suitable cationic exchange chromatography media include chromatography media such as carboxymethyl (CM), sulfopropyl (SP) or methyl sulfonate (S). In some embodiments, the cationic exchange chromatography resin is a SP sepharose fast flow resin.

In one embodiment the HNS obtained following the cationic exchange step is further filtered. In certain embodiments, the HNS is further filtered by, for example, diafiltration or ultrafiltration.

In one step, the purification occurs when the material containing the crude HNS is loaded onto a matrix and pre-equilibrated. The matrix is then washed to remove impurities. It is contemplated that column characteristics may be altered in bore size and length to allow elution with various gradients. As will be appreciated by one of skill in this art, the washing and elution solvents are determined by the matrix used and the polarity of the HNS in such an environment.

Extraction and/or purification of HNS from the bulk HNS composition from an anionic exchange chromatography resin can be optimized upon adjustment of pH levels. For example, a pH level of 7.0 has been shown to optimize extraction and purification. Accordingly, in certain embodiments, the pH of the unpurified bulk HNS composition is adjusted to a pH of about 7.0 prior to contacting the HNS with the anionic exchange chromatography resin. In certain embodiments, the material to be loaded on the anion exchange column is adjusted from about 50 mM to about 100 mM NaAcetate. In some embodiments, the solution containing the HNS composition to be loaded on the anionic exchange resin has a sodium acetate concentration from about 50 to about 100 mM. It has been determined that a conductivity of from about 3-4 mS/cm of the HNS composition facilitates the removal of high pI HNS species using anionic exchange chromatograph resins. Accordingly, in certain embodiments, the conductivity of the HNS composition is adjusted to obtain a conductivity of from about 3 to about 4 mS/cm prior to contacting the HNS composition with anionic exchange chromatography resin. In another embodiment, the conductivity is adjusted to about 3.5 mS/cm prior to contacting the HNS composition with the anionic exchange chromatography resin. In certain embodiments, the HNS composition is viral inactivated prior to loading on the anionic exchange column. In yet another embodiment, the HNS composition is filtered using a 0.2 μm filter prior to loading on the anionic exchange column.

In one embodiment, the anionic exchange column is washed with about 5 column volumes of a buffer containing about 20 mM MES-Tris and about 20 mM NaCl at a pH of about 7.0 prior to elution of the enriched HNS composition from the anion exchange column. In certain embodiments there are additional elution steps between contacts with each chromatography resin. In one embodiment, the HNS is eluted from the anionic exchange chromatography resin using a buffer constituting about 20 mM MES-Tris and about 180 mM NaCl at about pH 7.0. In certain embodiments, the percent recovery of the enriched HNS in the flow through and wash is measured by absorbance units, enzyme activity or ELISA. In one embodiment, the host cell protein clearance is about two fold after this step. In another embodiment, the process removes from about 10 to about 25% of a high pI HNS. In yet another embodiment, the removal of the high pI HNS leads to improved solubility.

In certain embodiments, the hydrophobic interaction resin is equilibrated with a buffer comprising about 20 mM MES-Tris and a NaCl concentration of about 1.1 to 1.5 M, at a pH of about 7.0 and a conductivity of from about 90 to about 120 mS/cm prior to contacting the HNS composition with the hydrophobic interaction column. Such concentrations, pH and conductivity facilitate the binding of HNS to the hydrophobic interaction column, thereby optimizing the purification of the HNS composition.

In certain embodiments, the eluate from the anionic exchange chromatography step containing enriched HNS is the starting material for the hydrophobic interaction step. In one embodiment, the NaCl concentration of the HNS composition is adjusted to achieve a NaCl concentration of from about 1.1 M to about 1.5 M NaCl prior to contacting the HNS composition with the hydrophobic interaction column. In another embodiment, the NaCl concentration is adjusted to about 1.2 M prior to contacting the HNS composition with the hydrophobic interaction column. The pH of the HNS composition is adjusted to about 7.0 prior to being contacted with the hydrophobic interaction column. In some embodiments, the HNS composition is adjusted to obtain a conductivity of from about 85 to 120 mS/cm at 25° C. prior to contacting the HNS composition with the hydrophobic interaction column. In some embodiments, the HNS composition is adjusted to obtain a conductivity of from about 90 to 110 mS/cm at 25° C. prior to contacting the HNS composition with the hydrophobic interaction column.

In certain embodiments, the HNS composition adsorbed to the hydrophobic interaction resin is washed with 4 column volumes of a buffer comprising about 20 mM MES-Tris to wash out impurities and a NaCl concentration of from about 1.1M to about 1.5M, at a pH of about 7.0. In yet another embodiment, the NaCl concentration is about 1.2M.

In one embodiment, the hydrophobic interaction column is eluted with about 4 column volumes of a buffer containing about 20 mM MES-Tris and about 180 to 220 mM NaCl at a pH of about 7.0 to elute the enriched HNS composition from the hydrophobic interaction column. In certain embodiments there are additional elution steps.

In one embodiment, the HNS is eluted from the hydrophobic interaction chromatography resin using a buffer constituting about 20 mM MES-Tris and about 200 mM NaCl at about pH 7.0 with a conductivity range from about 19 to about 23 mS/cm at 25° C. to optimize the recovery of purified HNS. In another embodiment, the pH range is from about 6.9 to 7.1. In certain embodiments, the percent recovery of the enriched HNS in the flow through and wash is measured by absorbance units, enzyme activity or ELISA. In one embodiment, the host cell protein clearance is about 35 to 45 fold after this step.

In certain embodiments, pooled eluates of enriched HNS obtained from the hydrophobic interaction column may be used as the starting material for purification employing a hydroxyapatite column. In some embodiments, the solution containing the HNS composition after elution from the hydrophobic interaction column is adjusted to a concentration of about 2 mM to about 4 mM of NaPO₄ to optimize purification. In certain embodiments, the concentration of NaPO4 is adjusted to about 2 mM and a pH of about 7.0+0.1. In one embodiment, the equilibration buffer contains about 20 mM MES-Tris and about 200 mM NaCl at about pH 7.0. In certain embodiments, the pH of the equilibration buffer is adjusted to from about 7.0 to about 7.2. In yet another embodiment, the HNS composition is filtered using a 0.2 μm filter prior to loading on the anionic exchange column. In another embodiment, the equilibration buffer contains about 2 mM NaPO₄, about 20 mM MES-Tris and about 200 mM NaCl at a pH of about 7.0.

In one embodiment, the hydroxyapatite column is washed with about 4 column volumes of a buffer containing about 2 mM to about 4 mM of NaPO4, about 20 mM MES-Tris and about 200 mM NaCl at a pH of from about 7.0 to about 7.2 prior to elution of the enriched HNS composition from the hydroxyapatite column. In another embodiment, the wash buffer contains about 2 mM NaPO₄, about 20 mM MES-Tris and about 200 mM NaCl at a pH of about 7.0.

In some embodiments, the HNS contacted with the hydroxyapatite column is eluted with a solution containing about 25 mM NaPO₄ at a pH of about 7.4 to about 7.6. In another embodiment, the HNS loaded onto the hydroxyapatite column is eluted with an eluent containing from about 20 mM NaPO₄ to about 30 mM NaPO₄ at a pH of about 7.0 to about 7.6. In one embodiment, the elution buffer contains about 20 mM NaPO₄, about 25 mM MES-Tris at a pH of about 7.5+0.1. In certain embodiments, the elution step may be repeated at least once. In certain embodiments, the percent recovery of the enriched HNS in the flow through and wash is measured by absorbance units, enzyme activity or ELISA.

In certain embodiments, pooled eluates of enriched HNS obtained from the hydroxyapatite column may be used as the starting material for purification employing a cationic exchange column. In yet another embodiment, the HNS composition in the starting material is adjusted to obtain a conductivity of about 3 to about 4 mS/cm prior to loading on the cationic exchange column to optimize binding of HNS to the cationic resin. In some embodiments, the conductivity is adjusted to about 3 mS/cm and the solution comprises about 20 mM sodium acetate at about pH 5.0 to optimize binding of HNS to the cationic column. In yet another embodiment, the conductivity of the HNS composition loaded on the cationic exchange resin is about 4 mS/cm and the solution contains about 40 mM sodium acetate at about pH 5.0 to optimize binding of HNS to the cationic column. In another embodiment, the conductivity of the HNS composition loaded on the cationic exchange resin is about 3.5 mS/cm+0.5 and the pH is about 5.0. In another embodiment, the HNS composition is filtered using a 0.2 μm filter prior to loading on the cationic exchange column.

In one embodiment, the equilibration buffer contains about 50 mM NaAcetate, from about 20 to about 40 mM NaCl and a pH of about 5.0. In certain embodiments, the pH of the equilibration buffer is adjusted to from about 4.9 to about 5.1. In another embodiment, the equilibration buffer contains about 50 mM NaAcetate, about 20 mM NaCl, a pH of about 5.0, and a conductivity range from about 5 to about 7 mS/cm.

In one embodiment, the cationic exchange column is washed with about 4 column volumes of a buffer containing about 50 mM NaAcetate, from about 20 mM to 40 mM NaCl at a pH of from about 5.0 to about 7.2 prior to elution of the enriched HNS composition from the cationic exchange column. In another embodiment, the wash buffer contains about 50 mM NaAcetate, about 20 mM NaCl, a pH of about 5.0, and a conductivity range from about 5 to about 7 mS/cm.

In some embodiments, the elution of the HNS from the cationic exchange resin is carried out with an eluent comprising about 50 mM sodium acetate and from about 90 mM to about 100 mM NaCl at a pH of about 4.9 to about 5.1. In certain embodiments, the elution of the HNS from the cationic exchange resin is carried out with an eluent comprising about 50 mM sodium acetate and about 90 mM NaCl, at a pH of about 5.0+0.1. In certain embodiments, the eluent has a conductivity range of from about 12 to about 14 mS/cm. In certain embodiments, the elution step may be repeated at least once. In certain embodiments, the percent recovery of the enriched HNS in the flow through and wash is measured by absorbance units, enzyme activity or ELISA.

Another embodiment described herein is a purified HNS which has been isolated by the methods above to a level of purity that is greater than about 90% free of contaminants. Preferably, the material is greater than 80%, 85%, 90%, 91%, 92%, 93%, 94%, 95%, 96%, 97%, 98%, or even greater than 99% free of contaminants. The degree of purity may be assessed by any suitable means known in the art.

Products and processes described herein can be useful for treating and/or preventing any disease/condition in a subject whereby glycosaminoglycans have been found to be important in the development and/or progression of the disease. Certain embodiments can be particularly useful for treating and/or preventing any disease or condition in a subject whereby HNS is either non-functional or absent. Treating a disease also includes exacting a desired improvement in the disease or symptoms of the disease.

The compositions disclosed herein may be used alone or in combination with another therapeutic agent for treating a disease associated with mucopolysaccharoidosis or its sequellae in a subject. These additional therapeutic agents can be administered prior to administration of the composition, or they can be administered at the same time or after administration of the composition. Subjects can be, for example, any human or non-human vertebrate, e.g., dog, cat, horse, cow, pig.

In one embodiment, the formulation buffers for the purified HNS compositions can be a phosphate buffer, such as 5 mM Sodium Phosphate, 145 mM NaCl, pH 7.0. Other suitable buffers are known to the skilled artisan.

In certain embodiments, the final HNS concentration is above 5 grams per liter, above 10 grams per liter, above 15 gram per liter, above 20 grams per liter.

Purified HNS compositions described herein may be administered topically (including ophthalmic and to mucous membranes including vaginal and rectal delivery), pulmonary (e.g., by inhalation or insufflation of powders or aerosols, including by nebulizer; intratracheally, intranasally), orally or parenterally. In certain embodiments parenteral administration is preferred and includes intravenous, intraarterial, subcutaneous, intraperitoneal intramuscular, intracranial, intrathecal or intraventricular, administration.

The embodiments described herein will be further illustrated by the following Examples, which should not be construed as limiting. The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or the entire group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists, (e.g., in Markush group or similar format) it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. The entire contents of all of the references (including literature references, issued patents and published patent applications and websites) cited throughout this application are hereby expressly incorporated by reference.

### EXEMPLIFICATION

### Example I

### Purification of Human HNS

The objective of the present studies was to obtain a large quantity of recombinant human HNS (with increased solubility). Stably transfected HT1080 cells were grown under bioreactor culture conditions, and active HNS enzyme was purified from the cell medium. The liquid chromatography apparatus used were the AKTA Explorer Chromatography System from GE Healthcare ((Piscataway, N.J.), Model: 18-1403-00) and the Genesys 6 UV-Vis Spectrophotometer from Thermo Scientific ((Waltham, Mass.), Catalog #335908000, Serial 2M6F078007). The following chromatography resins were employed: Q Sepharose Fast Flow from GE Healthcare ((Piscataway, N.J.), catalog #17-0510-04); Phenyl Sepharose 6 Fast Flow from GE Healthcare ((Piscataway, N.J.), catalog #17-0973-04); SP Sepharose Fast Flow from GE Healthcare ((Piscataway, N.J.), catalog #17-0729-04); and Ceramic Hydroxyapaptite, Type I, 80 μm particle size, from Bio-Rad ((Hercules, Calif.), catalog #157-0085).

The chromatography columns used in this example include Kontes 30×1.0 cm Column from Kontes Glas ((Vineland, N.J.), Catalog #420830-3000); XK 16/40 Column from GE Healthcare ((Piscataway, N.J.), catalog #18-8774-01); XK 5/30 Column from GE Healthcare ((Piscataway, N.J.), catalog #18-8751-01)); and Omnifit 10×25 mm Column from Bio-Chem Valve ((Boonton, N.J.), Catalog #006CC-10-02-AF).

A sandwich-based ELISA assay utilizing goat antibodies custom-generated by Cygnus Technologies against the HT1080 host cell lysates was used to determine the host cell protein concentration. Fifty microliters of samples diluted in sample diluent (20 mM sodium phosphate, 0.1% ProClin 300, pH 6.0), assay control (75 ng/ml) and standards are simultaneously incubated with 100 μl of HRP-conjugated antibody (1:95 dilution in conjugate diluent: Cygnus Technologies HRP Conjugate Diluent with 3 mg/ml normal goat IgG) on a pre-coated micro-ELISA plate for 2 hours at ambient temperature on a Titer Plate shaker.

Contents of the wells were removed at the end of incubation and the plate washed 4 times with ELISA wash buffer (25 mM Tris-HCl with 0.425% NaCl (w/v), 0.025% ProClin 300, pH 7.2). One hundred microliters of TMB substrate solution (tetramethyl benzediene; BioFx TMBW1000-01) was then added to each well and the plate incubated for another 30 minutes. The colorimetric reaction was terminated by the addition of 100 μl of stop solution (0.5 NH₂SO₄) and the absorbance at 450 nm measured using SPECTRAmax PLUS 384 Microplate Spectrophotometer with background subtraction set at 650 nm. A standard curve (0-200 ng/ml) was constructed using SoftMax 4.8 software and HCP concentration in the samples intrapolated. Using this assay, the amount of HCP originating from the HT1080 cell line in HNS samples was quantified.

TK1315 rabbit polyclonal antibody specific for HNS in-house IgG purified was coated at 5.0 ug/mL on a MaxiSorp Nunc Immuno plate for one hour at 37° C. After washing the plate three times with phosphate-buffered saline with 0.05% Tween 20 (PBST), the wells were blocked with 2% bovine serum albumin (BSA) in PBST. Samples and reference standards at appropriate dilution were incubated for one hour at 37° C. After washing the plate four times with PBST, the secondary antibody, TK1315 rabbit polyclonal antibody specific for HNS in-house IgG purified-HRP conjugate (1:3000) was applied. After incubation at 37° C. for 30 minutes, the plate was washed three times with PBST. TMB substrate (Bio-Rad, Hercules, Calif.) was applied, and the plate was incubated for 15 minutes at 37° C. before stopping the reaction with 2 M sulphuric acid. The plate was read at 450 nm, and a quadratic curve fit was used to generate the standard curve. This assay was used to quantify the amount of HNS in the samples. The concentration of the purified HNS protein was measured by A₂₈₀ absorbance using a Genesys 6 UV-Vis Spectrophotometer.

A two step HNS activity assay utilizing 4-methylumbelliferyl 2-sulfamino-2-deoxy-alpha-D-glucopyranoside as substrate was used to determine HNS activity. Ten microliters of samples diluted in substrate/reaction buffer was added to the assay plate (Costar 96 well plate, Corning #3912), followed by 20 μl of substrate solution (20 mM 4-methylumbelliferyl 2-sulfamino-2-deoxy-alpha-D-glucopyranoside in substrate/reaction buffer). The plate was incubated at 37° C. for one hour in Jitterbug (Boekel Scientific) with mixer setting at 1 for the initial 3 minutes. At the end of incubation, 6 μl of Pi/Ci stop buffer was added and the plate mixed for 3 minutes in Jitterbug with mixer setting at 1 to stop the first step of the reaction. Forty-six microliters of standards (0-50 uM 4-methylumbelliferone in substrate/reaction buffer; 0-2300 picomole) was then added to the first 2 blank rows of the plate followed by the addition of ten microliters of a-glucodidase (250 U per ml in 0.2% deactivated BSA) to the wells that contain samples and substrate/reaction buffer blanks (not standards). The plate was incubated at 37° C. for another hour in Jitterbug with mixer setting at 1 for the initial 3 minutes. At the end of incubation, 200 μl of Carbonate stop buffer (0.5 M sodium carbonate, pH 10.7, 0.025% Triton-100) was added to the wells that contain standards, samples and blanks to terminate the reaction.

The content of each well was mixed three times with the “shake plate” function using SpectraMax M2 multi-detection microplate reader and the fluorescence at 460 nm measured. A standard curve (0-2300 picomole) was constructed using Microsoft Excel and HNS activity in the samples intrapolated. One activity unit is defined as producing 1 picomole of 4-methylumbelliferone in one hour at 37° C. HNS activity was calculated and expressed as U/ml (or U/mg if the protein concentration is known). Using this assay, the activity values of HNS samples were quantified.

The process for purification consisted of using BioSepra™ MEP HyperCel sorbent (Pall Life Sciences, P/N #12035-036) capture to yield unpurified bulk material.

Prior to performing the Q column step, a viral inactivation process was performed by adding 1% Tween 80) and 0.3% TnBP to the unpurified bulk and holding the mixture at ambient for 3 to 16 hours. After viral inactivation, the mixture was filtered with a 0.2 um filter. Q load conductivity at 3.0, 3.5 and 4.0 mS/cm was studied. The operational conditions for the three runs are summarized in Table 1. The bulk load material for the Q runs was adjusted to 100 mM NaAcetate. The flow rate for the Q runs was 150 cm/hour.

TABLE 1
Operation Conditions for the Q Runs - Load Evaluation

| | | Q load at | Q load at |
|---|---|---|---|
| | | 3.0 mS/cm | 4.0 mS/cm |
| | Q load at | (low | (high |
| Process Steps | 3.5 mS/cm | conductivity) | conductivity) |
| Column Size* | 5.0 × 17.7 cm | 2.6 × 16.5 cm | 2.6 × 16.5 cm |
| Unpurified bulk | MEP Capture Eluates | | |
| (UPB) | | | |
| Sanitization | 0.5M NaOH | | |
| Equilibration | 20 mM MES-Tris, 20 mM NaCl, pH 7.0 | | |
| Load | Adjusted to | Adjusted to | Adjusted to |
| | 100 mM | 100 mM | 100 mM |
| | NaAcetate and | NaAcetate and | NaAcetate and |
| | pH 7. 0 | pH 7.0 | pH 7.0 |
| | viral | viral | viral |
| | inactivated | inactivated | inactivated |
| | adjusted | adjusted | adjusted |
| | conductivity | conductivity | conductivity |
| | to 3.5 mS/cm | to 3.0 mS/cm | to 4.0 mS/cm |
| | 0.2 μm | 0.2 μm | 0.2 μm |
| | filtered | filtered | filtered |
| | loaded at | loaded at | loaded at |
| | about 3 mg | about 3 mg | about 3 mg |
| | HNS/mL resin | HNS/mL resin | HNS/mL resin |

| | |
|---|---|
| Wash | 5 CV of 20 mM MES-Tris, 20 mM NaCl, pH 7.0 |
| Elute | 20 mM MES-Tris, 180 mM NaCl, pH 7.0 |
| | collect based on 100 mAU-50 mAU setpoints |
| Strip | 3 CV of 20 mM MES-Tris, 1M NaCl |
| Clean | 3 CV of 1M NaOH, 2M NaCl |

*The results from the 5 cm columns should be comparable to that from 2.6 cm column. The flow rate for the Q runs is 150 cm/hour.

The results from the Q runs are summarized in Table 2

TABLE 2
Results of the Q runs - Load evaluation

| | | FT/Wash + PrePeak (<100 mAU) | | |
|---|---|---|---|---|
| | Loading | % Recovery | % Recovery | % Recovery |
| | Condition | by AD | by Activity | by ELISA |
| Q (low | 3.0 mS/cm | 56.8 | 13.0 | 17.8 |
| conductivity) | | | | |
| Q (control) | 3.5 mS/cm | 54.9 | 13.4 | 20.1 |
| Q (high | 4.0 mS/cm | 49.1 | 13.9 | Sample not |
| conductivity) | | | | tested |

| | Eluate (100 mAU-50 mAU) | | | | |
|---|---|---|---|---|---|
| | Volume | % Recovery | % Recovery | % Recovery | HCP Fold |
| | (CV) | by AU | by Activity | by ELISA | Clearance |
| Q (low | 1.5 | 35.1 | 83.6 | 75.6 | 2 |
| conductivity) | | | | | |
| Q (control) | 1.6 | 40.4 | 70.0 | 79.9 | 2 |
| Q (high | 1.2 | 37.2 | 71.1 | 64.5 | 2 |
| conductivity) | | | | | |

The Q process was designed to remove 10-25% of high pI HNS to improve the solubility of HNS drug substance. Based on the results from the Q evaluation runs (Table 2), loading the Q column at 3.0, 3.5, and 4.0 mS/cm resulted in similar HNS loss in Q FT/Wash and similar HCP clearance of the eluate. There was some variation in eluate recovery by activity and ELISA, which may be caused by assay variations. It was expected that the recoveries would be similar for all three runs since there was similar HNS loss in the FT/wash. All recoveries by AU, activity, and ELISA were considered acceptable. Based on these results, the conductivity of the loading material was set to 3.5±0.5 mS/cm.

### Example 2

### Phenyl Sepharose Column

Phenyl loading at 1.1 M, 1.2 M, 1.3M, and 1.5M NaCl was studied. Phenyl elution at 180 mM, 200 mM, and 220 mM NaCl was also studied. The pH of the Phenyl load was not tested, as the HIC column is not expected to be sensitive to the pH changes in the loading process (pH 7.0±0.1). The operational conditions for the Phenyl runs are summarized in Tables 3 and 4. The flow rate for the Phenyl runs was 150 cm/hour.

TABLE 3
Operation Conditions of the Phenyl Runs - Load Evaluation

| | | Phenyl Load at | | |
|---|---|---|---|---|
| | Phenyl Load at | 1.2M NaCl | Phenyl Load at | Phenyl Load at |
| Process Steps | 1.1M NaCl | (Control) | 1.3M NaCl | 1.5M NaCl |
| Column Size | 1.6 × 17.7 cm | | | |
| Starting | Q Eluate | | | |
| Material | | | | |
| Sanitization | 0.5M NaOH | | | |
| Equilibration | 20 mM | 20 mM | 20 mM | 20 mM |
| | MES-Tris, | MES-Tris, | MES-Tris, | MES-Tris, |
| | 1.1M NaCl, | 1.2M NaCl, | 1.3M NaCl, | 1.5M NaCl, |
| | pH 7.0 | pH 7.0 | pH 7.0 | pH 7.0 |
| Load | Adjusted Q | Adjusted Q | Adjusted Q | Adjusted Q |
| | eluate to | eluate to | eluate to | eluate to |
| | 1.1M NaCl | 1.2M NaCl | 1.3M NaCl | 1.5M NaCl |
| | and pH 7.0 | and pH 7.0 | and pH 7.0 | and pH 7.0 |
| | 0.2 μm | 0.2 μm | 0.2 μm | 0.2 μm |
| | filtered | filtered | filtered | filtered |
| | loaded at | loaded at | loaded at | loaded at |
| | 5.7 AU/mL | 5.7 AU/mL | 5.7 AU/mL | 5.7 AU/mL |
| | resin | resin | resin | resin |
| Wash | 4 CV of 20 mM | 4 CV of 20 mM | 4 CV of 20 mM | 4 CV of 20 mM |
| | MES-Tris, | MES-Tris, | MES-Tris, | MES-Tris, |
| | 1.1M NaCl, | 1.2M NaCl, | 1.3M NaCl, | 1.5M NaCl, |
| | pH 7.0 | pH 7.0 | pH 7.0 | pH 7.0 |

| | |
|---|---|
| Elute | 20 mM MES-Tris, 200 mM NaCl, pH 7.0 |
| | collect based on 100 mAU-50 mAU setpoints |
| Strip 1 | 3 CV of water |
| Strip 2 | 2 CV of 20% ethanol |
| Clean | 2 CV of 0.5M NaOH |

The flow rate for the Phenyl runs is at 150 cm/hour.

TABLE 4
Operation Conditions of the Phenyl Runs - Elution Evaluation

| | | Phenyl elution | |
|---|---|---|---|
| | Phenyl elution | at 200 mM NaCl | Phenyl elution |
| Process Steps | at 180 mM NaCl | (Control) | at 220 mM NaCl |
| Column Size | 1.6 × 17.7 cm | | |
| Starting | Q Eluate | | |
| Material | | | |
| Sanitization | 0.5M NaOH | | |
| Equilibration | 20 mM MES-Tris, 1.2M NaCl, pH 7.0 | | |
| Load | Adjusted Q Eluate to 1.2M NaCl and pH 7.0 | | |
| | 0.2 μm filtered | | |
| | Loaded at 5.7 AU/mL resin | | |
| Wash | 4 CV of 20 mM MES-Tris, 1.2M NaCl, pH 7.0 | | |
| Elute | 20 mM MES-Tris, | 20 mM MES-Tris, | 20 mM MES-Tris, |
| | 180 mM NaCl, | 200 mM NaCl, | 220 mM NaCl, |
| | pH 7.0 | pH 7.0 | pH 7.0 |
| | Collect | Collect | Collect |
| | based on | based on | based on |
| | 100 mAU-50 mAU | 100 mAU-50 mAU | 100 mAU-50 mAU |
| | setpoints | setpoints | setpoints |

| | |
|---|---|
| Strip 1 | 3 CV of water |
| Strip 2 | 2 CV of 20% ethanol |
| Clean | 2 CV of 0.5M NaOH |

The flow rate for the Phenyl runs is at 150 cm/hour.

The results from the Phenyl runs are summarized in Tables 5 and 6.

TABLE 5
Results of the Phenyl Runs - Load Evaluation

| | | FT/Wash + PrePeak (<100 mAU) | | |
|---|---|---|---|---|
| | | % | | |
| | Conductivity | Recovery | % Recovery | % Recovery |
| | (mS/cm) | by AU | by Activity | by ELISA |
| Phenyl (1.1M | Buffer: 96.0 | 3.3 | LOQ* | 0.4 |
| NaCl) | Load: 89.8 | | | |
| Phenyl (1.2M | Buffer: 104.2 | 2.3 | LOQ* | 0.1 |
| NaCl) | Load: 97.9 | | | |
| Phenyl (1.3M | Buffer: 110.3 | 2.1 | LOQ* | 0.0 |
| NaCl) | Load: 103.7 | | | |
| Phenyl (1.5M | Buffer: 123.1 | 1.7 | LOQ* | 0.0 |
| NaCl) | Load: 116.5 | | | |

| | Eluate (100 mAU-50 mAU) | | | | | |
|---|---|---|---|---|---|---|
| | | | % | | | |
| | Vol- | % | Recovery | % | Total | |
| | ume | Recovery | by | Recovery | HCP | HCP Fold |
| | (CV) | by AU | Activity | by ELISA | (ng) | Clearance |
| Phenyl | 5.8 | 83.0 | 85.6 | 80.4 | 215186 | 42 |
| (1.1M | | | | | | |
| NaCl) | | | | | | |
| Phenyl | 5.9 | 83.9 | 87.0 | 87.3 | 205548 | 45 |
| (1.2M | | | | | | |
| NaCl) | | | | | | |
| Phenyl | 5.9 | 83.9 | 88.2 | 87.8 | 271155 | 36 |
| (1.3M | | | | | | |
| NaCl) | | | | | | |
| Phenyl | 6.0 | 85.0 | 90.4 | 89.7 | 242555 | 40 |
| (1.5M | | | | | | |
| NaCl) | | | | | | |

*LOQ is equivalent to <150 U/mL

TABLE 6
Results of the Phenyl Runs -Elution Evalaution

| | Buffer | Eluate (100 mAU-50 mAU) | | | | | |
|---|---|---|---|---|---|---|---|
| | Conductivity | Volume | % Recovery by | % Recovery by | % Recovery by | Total | HCP Fold |
| | (mS/cm) | (CV) | AU | Activity | ELISA | HCP (ng) | Clearance |
| Phenyl | 119.5 | 5.7 | 82.9 | 87.2 | 88.1 | 202367 | 45 |
| (180 mM | | | | | | | |
| NaCl) | | | | | | | |
| Phenyl | 21.0 | 5.9 | 83.9 | 87.0 | 87.3 | 205548 | 45 |
| (200 mM | | | | | | | |
| NaCl) | | | | | | | |
| Phenyl | 22.9 | 6.0 | 83.4 | 85.3 | 86.7 | 220549 | 42 |
| (220 mM | | | | | | | |
| NaCl) | | | | | | | |

Loading at 1.1 M, 1.2 M, 1.3 M, and 1.5 M NaCl, pH 7.0 (conductivity range from 90 to 117 mS/cm at 25° C.), resulted in similar HNS loss in FT/Wash, elution volumes, recoveries, and HCP clearance of the Phenyl eluate (Table 5). Elution of the Phenyl column, at 180 mM, 200 mM, and 220 mM NaCl, pH 7.0 (conductivity range from 20 to 23 mS/cm at 25° C.), resulted in similar elution volumes, recoveries, and HCP clearance of the Phenyl eluate (Table 6).

Based on these results, adjusting the Phenyl load to 1.2 M NaCl, pH 7.0±1.0 with a conductivity range of 90 to 110 mS/cm at 25° C. may be preferred. A recommended Phenyl equilibration and wash buffer is 20 mM MES-Tris, 1.2 M NaCl, pH 7.0±1.0 with a conductivity range of 100 to 120 mS/cm at 25° C. A recommended Phenyl elution buffer is 20 mM MES-Tris, 200 mM NaCl, pH 7.0±1.0 with a conductivity range of 19 to 23 mS/cm at 25° C.

### Example 3

### Ceramic Hydroxyapatite (HA) Column

HA loading and elution of a different sodium phosphate concentrations and different pH values were studied. The operational conditions for the experimental runs are summarized in Tables 7 and 8. The flow rate for the HA runs was at 150 cm/hr.

TABLE 7
Operation Conditions of the HA Runs - Load Evaluation

| | HA Load at | HA Load at | HA Load at |
|---|---|---|---|
| | 2 mM NaPO₄, | 2 mM NaPO₄, | 2 mM NaPO₄, |
| | pH 7.0 | pH 7.0 | pH 7.0 |
| Process Steps | (Control) | (Control) | (Control) |
| Column Size | 1.0 × 24.5 cm | | |
| Starting | Phenyl Eluate Pool (Phenyl 1-Phenyl 6) | | |
| Material | | | |
| Sanitization | 0.5M NaOH | | |
| Equilibration | 2 mM NaPO₄ | 4 mM NaPO₄ | 2 mM NaPO₄ |
| | 20 mM MES-Tris, | 20 mM MES-Tris, | 20 mM MES-Tris, |
| | 200 mM NaCl, | 200 mM NaCl, | 200 mM NaCl, |
| | pH 7.0 | pH 7.0 | pH 7.2 |
| Load | Adjusted | Adjusted | Adjusted |
| | Phenyl pool to | Phenyl pool to | Phenyl pool to |
| | 2 mM NaPO₄ | 4 mM NaPO₄ | 2 mM NaPO₄ and |
| | 0.2 μm | 0.2 μm | pH 7.2 |
| | filtered | filtered | 0.2 μm |
| | Loaded at | Loaded at | filtered |
| | 7.6 AU/mL | 7.6 AU/mL | Loaded at |
| | resin | resin | 7.6 AU/mL |
| | | | resin |
| Wash | 4 CV of | 4 CV of | 4 CV of |
| | 2 mM NaPO₄ | 4 mM NaPO₄ | 2 mM NaPO₄ |
| | 20 mM MES-Tris, | 20 mM MES-Tris, | 20 mM MES-Tris, |
| | 200 mM NaCl, | 200 mM NaCl, | 200 mM NaCl, |
| | pH 7.0 | pH 7.0 | pH 7.2 |

| | |
|---|---|
| Elute | 25 mM NaPO₄, pH 7.0 |
| | Collect based on 100 mAU-50 mAU setpoints |
| Strip | 2.5 CV of 250 mM NaPO₄, pH 7.0 |
| Clean | 2 CV of 0.5M NaOH |

Flowrate for the HA runs is at 150 cm/hour

TABLE 8
Operation Conditions of the HA Runs - Elution evaluation

| | HA elution at | | | | |
|---|---|---|---|---|---|
| | 25 mM PO₄, | HA elution at | HA elution at | HA elution at | HA elution at |
| | pH 7.5 | 20 mM PO₄, | 30 mM PO₄, | 25 mM PO₄, | 25 mM PO₄, |
| Process Steps | (Control) | pH 7.5 | pH 7.5 | pH 7.4 | pH 7.6 |
| Column Size | 1.0 cm × 24.5 cm | | | | |
| Starting | Phenyl Eluate Pool (Phenyl 1-Phenyl6) | | | | |
| Material | | | | | |
| Sanitization | 0.5M NaOH | | | | |
| Equilibration | 2 mM NaPO₄ 20 mM MES-Tris, 200 mM NaCl, pH 7.0 | | | | |
| Load | Adjusted Phenyl Pool to 2 mM NaPO₄ | | | | |
| | 0.2 μm filtered | | | | |
| | Loaded at 7.67 AU/mL resin | | | | |
| Wash | 4 CV 2 mM NaPO₄ 20 mM MES-Tris, 200 mM NaCl, pH 7.0 | | | | |
| Elute | 25 mM NaPO₄, | 20 mM NaPO₄, | 30 mM NaPO₄, | 25 mM NaPO₄, | 25 mM NaPO₄, |
| | pH 7.5 | pH 7.5 | pH 7.5 | pH 7.4 | pH 7.6 |
| | Collect | Collect | Collect | Collect | Collect |
| | based on | based on | based on | based on | based on |
| | 100 mAU-50 mAU | 100 mAU-50 mAU | 100 mAU-50 mAU | 100 mAU-50 mAU | 100 mAU-50 mAU |
| | setpoints | setpoints | setpoints | setpoints | setpoints |

| | |
|---|---|
| Strip | 2.5 CV of 250 mM NaPO₄, pH 7.0 |
| Clean | 2 CV of 0.5M NaOH |

### Results

The results from the HA runs are summarized in Tables 9 and 10. The level of quantitation (LOC) is equivalent to <150 U/ml.

TABLE 9
Results of the HA Runs - 3 Load Evaluation

| | FT/Wash + PrePeak (<100 mAU) | | | |
|---|---|---|---|---|
| | Conductivity | % Recovery | % Recovery | % Recovery |
| | (mS/cm) | by AU | by Activity | by ELISA |
| HA | Buffer: 21.8 | 3.3 | LOQ* | 2.1 |
| 2 mM NaPO₄, | Load: 22.7 | | | |
| pH 7.0 | | | | |
| HA 4 mM | Buffer: 21.9 | 7.4 | 5.3 | 5.3 |
| NaPO₄, pH | Load: 22.8 | | | |
| 7.0 | | | | |
| HA | Buffer: 21.8 | 3.7 | 2.2 | 2.7 |
| 2 mM NaPO₄, | Load: 22.8 | | | |
| pH 7.2 | | | | |

| | Eluate (100 mAU-50 mAU) | | | | | |
|---|---|---|---|---|---|---|
| | | | % | | | |
| | Vol- | % | Recovery | % | Total | |
| | ume | Recovery | by | Recovery | HCP | HCP Fold |
| | (CV) | by AU | Activity | by ELISA | (ng) | Clearance |
| HA | 4.1 | 81.4 | 84.9 | 89.6 | 8001 | 19 |
| 2 mM | | | | | | |
| NaPO₄, | | | | | | |
| pH 7.0 | | | | | | |
| HA | 4.3 | 76.5 | 79.8 | 86.3 | 13349 | 10 |
| 4 mM | | | | | | |
| NaPO₄, | | | | | | |
| pH 7.0 | | | | | | |
| HA | 3.9 | 80.6 | 74.1 | 84.0 | 26472 | 4 |
| 2 mM | | | | | | |
| NaPO₄, | | | | | | |
| pH 7.2 | | | | | | |

*LOQ is equivalent to <150 U/mL

TABLE 10
Results of the HA Runs - Elution Evaluation

| | | Eluate (100 mAU-50 mAU) | | | | | |
|---|---|---|---|---|---|---|---|
| | | | | % | | | |
| | Buffer | | % | Recovery | | Total | |
| | conductivity | Volume | Recovery | by | | HCP | HCP Fold |
| | (mS/cm) | (CV) | by AU | Activity | 78.0 | (ng) | Clearance |
| HA | 3.18 | 4.6 | 75.9 | 78.1 | 88.1 | 7806 | 19 |
| 20 mM | | | | | | | |
| NaPO₄, | | | | | | | |
| pH 7.5 | | | | | | | |
| HA 25 mM | 3.88 | 4.1 | 81.4 | 84.9 | 89.6 | 8001 | 19 |
| NaPO₄, | | | | | | | |
| pH 7.5 | | | | | | | |
| HA | 4.59 | 4.0 | 81.8 | 83.8 | 91.5 | 17654 | 8 |
| 30 mM | | | | | | | |
| NaPO₄, | | | | | | | |
| pH 7.5 | | | | | | | |
| HA | 3.79 | 4.5 | 78.7 | 81.2 | 82.3 | 11834 | 13 |
| 25 mM | | | | | | | |
| NaPO₄, | | | | | | | |
| pH 7.4 | | | | | | | |
| HA | 3.96 | 4.5 | 80.6 | 84.2 | 89.8 | 18429 | 8 |
| 25 mM | | | | | | | |
| NaPO₄, | | | | | | | |
| pH 7.6 | | | | | | | |

Loading the HA column at 4 mM NaPO₄ resulted in higher percent loss in absorbance units in the FT/Wash and lower percent recover by absorbance in the eluate (Table 9). At the HA state, the purity of the HA eluate is above 95% and recovery by absorbance units is more reliable than recovery based on activity or ELISA results, due to the relatively large variability in these assays. Loading the HA column at 2 mM NaPO₄, pH 7.2 resulted in lower HCP clearance in the eluate. Based on these results, the HA process appeared to be sensitive to phosphate concentration and pH. Adjusting the HA load to 2 mM NaPO₄, pH 7.0±0.1 may be preferred. HA equilibration and wash buffer of 2 mM NaPO₄, 20 mM MES-Tris, 200 mM NaCl, pH 7.0±0.1 also may be preferred.

Eluting the HA column with a 20 mM NaPO₄, pH 7.5 buffer resulted in lower percent recovery as measured by absorbance units (Table 10). Increasing phosphate concentration to 30 mM increased the percent recovery but reduced the HCP clearance in the eluate. It was also noticed that changing the pH of the elution buffer will also reduce the HCP clearance in the eluates. A recommended HA elution buffer is 20 mM NaPO₄, pH 7.5±0.1.

### Example 4

### SP Sepharose Column

SP loads at 3.0 and 4.0 mS/cm and SP EQ/Wash at 20 mM NaCl and 40 mM NaCl were studied. SP elution at different salt concentrations (80 mM NaCl, 90 mM NaCl, 100 mM NaCl) and different pH values (pH 4.9, pH 5.0, and pH 5.1) were also studied. The operational conditions for the experimental runs are summarized in Tables 11 and 12. The flow rate for the SP runs was 150 cm/hour.

TABLE 11
Operation Conditions of the SP Runs - Load Evaluation

| | SP load at | SP load at |
|---|---|---|
| | 4.0 mS/cm | 3.0 mS/cm |
| | (undiluted) and | (Control) and |
| | EQ/Wash at | EQ/Wash at |
| Process Steps | 40 mM NaCl | 20 mM NaCl |
| Column Size | 1.0 cm × 17.8 cm | |
| Starting | HA Eluate Pool (HA1-HA7) | |
| Material | | |
| Sanitization | 0.5M NaOH | |
| Equilibration | 50 mM NaAcetate, | 50 mM NaAcetate, |
| | 40 mM NaCl, | 20 mM NaCl, |
| | pH 5.0 | pH 5.0 |
| Load | Adjusted pH to 5.0 | Adjusted pH to 5.0 |
| | 0.2 μm filtered | and conductivity |
| | loaded with | to 3.0 mS/cm |
| | 8.5 mAU/mL | 0.2 μm filtered |
| | resin | loaded with |
| | | 8.5 mAU/mL |
| | | resin |
| Wash | 4 CV of 50 mM | 4 CV of 50 mM |
| | NaAcetate, | NaAcetate, |
| | 40 mM NaCl, | 20 mM NaCl, |
| | pH 5.0 | pH 5.0 |

| | |
|---|---|
| Elute | 50 mM NaAcetate, 90 mM NaCl, pH 5.0 |
| | Collect based on 50 mAU-50 mAU setpoints |
| Strip | 2.5 CV of 50 mM NaAcetate, 1M NaCl, pH 5.0 |
| Clean | 2 CV of 0.5M NaOH |

The flowrate for SP runs is at 150 cm/hour.

TABLE 12
Operation Conditions of the SP runs -Elution Evaluation

| | SP elution | | | | |
|---|---|---|---|---|---|
| | 90 mM NaCl, | SP elution | SP elution | SP elution | SP elution |
| | pH 5.0 | 80 mM NaCl, | 100 mM NaCl, | 90 mM NaCl, | 90 mM NaCl, |
| Process Steps | (Control) | pH 5.0 | pH 5.0 | pH 4.5 | pH 5.1 |
| Column Size | 1.0 cm × 17.8 cm | | | | |
| Starting | HA Eluate Pool (HA1-HA7) | | | | |
| Material | | | | | |
| Sanitization | 0.5M NaOH | | | | |
| Equilibration | 50 mM NaAcetate, 20 mM NaCl, pH 5.0 | | | | |
| Load | Adjusted pH to 5.0 and conductivity to 3.0 mS/cm | | | | |
| | 0.2 μm filtered | | | | |
| | loaded with 8.5 mAU/mL resin | | | | |
| Wash | 4 CV of 50 mM NaAcetate, 20 mM NaCl, pH 5.0 | | | | |
| Elute | 50 mM | 50 mM | 50 mM | 50 mM | 50 mM |
| | NaAcetate, | NaAcetate, | NaAcetate, | NaAcetate, | NaAcetate, |
| | 90 mM NaCl, | 80 mM NaCl, | 100 mM NaCl, | 90 mM NaCl, | 40 mM NaCl, |
| | pH 5.0 | pH 5.0 | pH 5.0 | pH 4.9 | pH 5.1 |
| | Collect | Collect | Collect | Collect | Collect |
| | based on | based on | based on | based on | based on |
| | 50 mAU-50 mAU | 50 mAU-50 mAU | 50 mAU-50 mAU | 50 mAU-50 mAU | 50 mAU-50 mAU |
| | setpoints | setpoints | setpoints | setpoints | setpoints |

| | |
|---|---|
| Strip | 2.5 CV of 50 mM NaAcetate, 1M NaCl, pH 5.0 |
| Clean | 2 CV of 0.5M NaOH |

The flowrate for SP runs is at 150 cm/hour.

The results from the SP runs are summarized in Tables 13 and 14.

TABLE 13
Results of the SP runs - Load evaluation

| | | FT/Wash + | |
|---|---|---|---|
| | | PrePeak (<100 mAU) | |
| | Conductivity | % Recovery | % Recovery |
| | (mS/cm) | by AU | by Activity |
| SP (pH 5.0 and | Buffer: 50.4 | 0.2 | LOQ* |
| diluted to | Load: 3.00 | | |
| 3.0 mS/cm)† | | | |
| SP (pH 5.0 and at | Buffer: 7.23 | 5.0 | 1.1** |
| 4.0 mS/cm)‡ | Load: 4.08 | | |

| | Eluate (100 mAU-50 mAU) | | | | |
|---|---|---|---|---|---|
| | Volume | % Recovery | % Recovery | Total | HCP Fold |
| | (CV) | by AU | by Activity | HCP (ng) | Clearance |
| SP (pH 5.0 and | 2.3 | 94.3 | 97.9 | 5620 | 4 |
| diluted to 3.0 | | | | | |
| mS/cm)† | | | | | |
| SP (pH 5.0 and at | 2.6 | 90.2 | 98.2** | 3479 | 4 |
| 4.0 mS/cm)‡ | | | | | |

†The SP column was loaded at 3.0 mS/cm and the EQ/Wash at 50 mM NaAcetate, 20 mM NaCl, pH 5.0.
‡The SP column was loaded at 4.0 mS/cm and the EQ/Wash at 50 mM NaAcetate, 40 mM NaCl, pH 5.0.
*LOQ is equivalent to <150 U/mL
**Note:
Yields were normalized.

TABLE 14
Results of the SP Runs -Elution Evaluation

| | | Eluate (100 mAU-50 mAU) | | | | |
|---|---|---|---|---|---|---|
| | Buffer | | | % | | |
| | con- | Vol- | % Re- | Recovery | Total | |
| | ductivity | ume | covery | by | HCP | HCP Fold |
| | (mS/cm) | (CV) | by AU | Activity | (ng) | Clearance |
| SP (80 mM | 11.5 | 4.7 | 90.0 | 90.8 | 4684 | 5 |
| NaCl, | | | | | | |
| pH 5.0) | | | | | | |
| SP (90 mM | 12.7 | 2.3 | 94.3 | 97.9 | 5620 | 4 |
| NaCl, | | | | | | |
| pH 5.0) | | | | | | |
| SP (100 mM | 13.7 | 1.4 | 95.6 | 95.3 | 4167 | 5 |
| NaCl, | | | | | | |
| pH 5.0) | | | | | | |
| SP (90 mM | 12.5 | 4.4 | 89.3 | 89.8 | 4088 | 5 |
| NaCl, | | | | | | |
| pH 4.9) | | | | | | |
| SP (90 mM | 12.8 | 1.3 | 95.3 | 97.7 | 4369 | 5 |
| NaCl, | | | | | | |
| pH 5.1) | | | | | | |

Loading the SP column with loads under diluted or undiluted conditions (conductivity range from 3.0 to 4.0 mS/cm) resulted in similar recoveries and HCP clearance of the SP eluate (Table 13). Washing the SP column at 20 mM or 40 mM NaCl, pH 5.0 (conductivity range from 5.0 to 7.2 mS/cm at 25° C.) resulted in similar recoveries and HCP clearance of the SP eluate (Table 13). Based on these results, adjusting SP load to pH 5.0±0.1 with a conductivity range of 3.5±0.5 mS/cm for loading may be preferred. A recommended SP equilibration and wash buffer is 50 mM NaAcetate, 20 mM NaCl, pH 5.0±0.1 with a conductivity range of 5 to 7 mS/cm.

Eluting the SP column at 80 mM, 90 mM, and 100 mM NaCl, pH 5.0 (conductivity range from 11.5 to 13.7 mS/cm at 25° C.) resulted in similar recoveries and HOP clearance (Table 14). However, the column volume of the eluate at 80 mM NaCl was 4.7 CV compared to 2.3 CV at 90 mM NaCl. Also, eluting the SP column at 90 mM NaCl, pH 4.9, pH 5.0, and pH 5.1 resulted in similar recoveries and HCP clearance. However, the eluate volume of the pH 4.9 elution was 4.4 CV compared to 2.3 CV of the eluate at pH 5.0. In both cases, the increase in volume of the eluate was due to tailing of the elution profile. To avoid collecting an excessive peak tail, it is recommended to collect the eluate from 50 mAU to 50 mAU or to a maximum of 3 CV, whichever comes first. A recommended SP elution buffer is 50 mM NaAcetate, 90 mM NaCl, pH 5.0+0.1 with a conductivity range of 12 to 14 mS/cm and peak collection of the eluate from 50 mAU to 50 mAU or to a maximum of 3 CV, whichever comes first.

These assays indicate that the protocols described above for preparing recombinant lysosomal sulfatase enzymes provide an efficient method for production of large quantities of highly purified enzyme, human Heparan-N-sulfatase (HNS).

What is claimed is:

1. A process for purifying heparan-N-sulfatase comprising the steps of:
a) contacting a heparan-N-sulfatase composition with an anionic exchange chromatography resin under conditions in which the heparan-N-sulfatase is adsorbed;
b) eluting the adsorbed heparan-N-sulfatase from the anionic exchange chromatography resin;
c) contacting the heparan-N-sulfatase composition obtained from step b) with a hydrophobic interaction chromatography resin;
d) eluting the adsorbed heparan-N-sulfatase from the hydrophobic interaction chromatography resin;
e) contacting the heparan-N-sulfatase composition obtained from step d) with a hydroxyapatite chromatography resin;
f) eluting the adsorbed heparan-N-sulfatase from the hydroxyapatite chromatography resin;
g) contacting the heparan-N-sulfatase composition obtained from step f) with a cationicexchange chromatography resin; and
h) eluting the adsorbed heparan-N-sulfatase from the cationic exchange chromatography resin.

2. A process according to claim 1, further comprising the step of:
contacting a heparan-N-sulfatase composition with a cellulose matrix linked to 4-mercapto-ethyl-pyridine.

3. A process according to claim 1, further comprising the step of:
inactivating virus in the heparan-N-sulfatase composition.

4. A process according to claim 1, wherein the anionic exchange chromatography resin of step a) is a Q sepharose fast flow resin.

5. A process according to claim 1, wherein the hydrophobic interaction chromatography resin of step c) is a phenyl sepharose 6 fast flow resin.

6. A process according to claim 1, wherein the hydroxyapatite chromatography resin of step e) is a ceramic hydroxyapatite type I resin.

7. A process according to claim 1, wherein the cationic exchange chromatography resin of step g) is a SP sepharose fast flow resin.

8. A process according to claim 1, further comprising the step of filtering the heparan-N-sulfatase.

9. A process according to claim 8, wherein filtration step is performed by diafiltration or ultrafiltration.

10. A process according to claim 1, further comprising the step of adjusting the heparan-N-sulfatase composition to a pH of about 7.0 prior to the performance of step a).

11. A process according to claim 10, wherein the solution has a sodium acetate concentration from about 50 to about 100 mM.

12. A process according to claim 1, further comprising the step of:
adjusting the heparan-N-sulfatase composition to obtain a conductivity of from about 3 to about 4 mS/cm prior to the performance of step a).

13. A process according to claim 1, wherein an eluent of step b) comprises about 20 mM MES-Tris and about 180 mM NaCl at about pH 7.0.

14. A process according to claim 1, further comprising the step of:
adjusting the heparan-N-sulfatase composition to achieve a NaCl concentration of from about 1.1M to about 1.5 M NaCl prior to the performance of step c).

15. A process according to claim 14, wherein the NaCl concentration is about 1.2 M.

16. A process according to claim 14, further comprising the step of:
adjusting the composition to obtain a conductivity of from about 90 to 110 mS/cm at 25° C.

17. A process according to claim 1, further comprising the step of:
equilibrating the resin prior to the performance of step c) with a buffer comprising about 20 mM MES-Tris and a NaCl concentration of about 1.2 M, at a pH of about 7.0 and a conductivity of from about 100 to about 120 mS/cm.

18. A process according to claim 1, further comprising the step of:
equilibrating the adsorbed heparan-N-sulfatase composition of step c) with an eluent comprising about 20 mM MES-Tris and a NaCl concentration of from about 180 mM to about 220 mM, at a pH of about 7.0.

19. A process according to claim 18, wherein the NaCl concentration is about 200 mM.

20. A process according to claim 1, further comprising the step of:
adjusting a solution containing the heparan-N-sulfatase composition obtained in step d) to a concentration of about 2 mM to about 4 mM of NaPO₄ prior the performance of step e).

21. A process according to claim 20, wherein the concentration of NaPO₄ is adjusted to about 2 mM.

22. A process according to claim 1, further comprising the step of:
eluting the heparan-N-sulfatase from the resin in step e) with an eluent comprising 25 mM NaPO₄ at a pH of about 7.5.

23. A process according to claim 1, further comprising the step of:
adjusting the heparan-N-sulfatase composition obtained in step f) to obtain a conductivity of about 3 to about 4 mS/cm prior to performing step g).

24. A process according to claim 23, wherein the conductivity is about 3 mS/cm and the solution comprises about 20 mM sodium acetate at about pH 5.0.

25. A process according to claim 24, wherein the conductivity is about 4 mS/cm and the solution contains about 40 mM sodium acetate at about pH 5.0.

26. A process according to claim 1, wherein step h) is carried out with an eluent comprising about 50 mM sodium acetate and about 90 mM NaCl at a pH of about 5.

27. A process according to claim 26, wherein the eluent has a conductivity of from about 12 to about 14 mS/cm.
